(19) United States
(12) Patent Application Publication
(10) Pub. No.: US 2003/0175958 A1
(43) Pub. Date: Sep. 18, 2003

(54) Methods for using bag expression as a cell differentiation agent and marker

(76) Inventors: John C. Reed, Rancho Santa Fe, CA (US); Pawel Kermer, San Diego, CA (US); Stanislaw Krajewski, San Diego, CA (US)

Correspondence Address: PERKINS COIE LLP, 101 Jefferson Drive, Menlo Park, CA, 94025-1114, US

(21) Appl. No.: 10/099,553

(22) Filed: Mar. 15, 2002

(51) Int. Cl. C12N 5/08; C12N 5/06; C12N 15/85

(52) U.S. Cl. 435/368; 435/325; 435/455

(57) ABSTRACT

The invention provides a method for promoting cell differentiation, which involves modifying a cell to increase expression of a BAG polypeptide that promotes differentiation of a cell, such as a neuronal cell, stem cell or neural progenitor cell. The invention provides another method for promoting cell differentiation, which involves modifying a cell to increase the amount of a nuclear localized BAG polypeptide, when the nuclear localized BAG polypeptide promotes differentiation of the cell. The invention also provides methods for reducing the rate of cell proliferation and suppressing apoptosis. The methods involve modifying a cell to increase the amount of a nuclear localized BAG polypeptide, when the nuclear localized BAG polypeptide inhibits proliferation, or suppresses apoptosis, respectively.

This invention was made with government support under grant numbers NS36821 and CA67329 awarded by the National Institutes of Health. The United States Government has certain rights in this invention.

### BACKGROUND OF THE INVENTION

Cellular homeostasis is the balance of cell proliferation and cell death that determines the life cycle of a cell. Both cellular proliferation and apoptosis, or programed cell death, are tightly regulated processes subject to numerous positive and negative signals. Perturbation of these signals can result in abnormally increased apoptosis or abnormally increased cell proliferation or survival, which contribute to the pathogenesis of human diseases, including autoimmune disorders, neurodegenerative process and cancer.

Dysregulation of cell death or cell proliferation is implicated in a wide variety of neurodegenerative processes, including Huntington's disease, Alzheimer's disease and Parkinson's disease. For many of these neurodegenerative diseases, there exist no effective therapies or cures. For example, untreated Parkinson's disease is a progressive and ultimately fatal neurodegenerative disorder characterized by loss of the pigmented dopaminergic neurons of the substantia nigra. The symptoms of Parkinson's disease can often be managed initially by administration of L-DOPA, the immediate precursor of dopamine. However, reduced efficacy of L-DOPA treatment typically occurs over time.

In Alzheimer's disease, the most common neurodegenerative disease and most frequent cause of dementia, progressive failure of memory and degeneration of temporal and parietal association cortex result in speech impairment and loss of coordination, and, in some cases, emotionally disturbance. Alzheimer's disease generally progresses over many years, with patients gradually becoming immobile, emaciated and susceptible to pneumonia.

Neuronal cell transplantation is a promising treatment modality for a variety of serious neurodegenerative diseases for which no effective therapeutic course exists, including Parkinson's disease and Alzheimer's disease as well as Huntington's disease, amyotrophic lateral sclerosis, multiple sclerosis, epilepsy and pain. However, the expansion of stem and precursor cell populations currently does not produce a cell population useful for therapeutic transplantation, since a relatively small number of neurons is produced, and even a smaller number survive and express the neuronal phenotype when grafted into the central nervous system.

Thus, there exists a need for therapeutic methods for treating neurological disorders, including methods for producing large numbers of neuronal cells useful for central nervous system transplantation. The present invention satisfies this need and provides related advantages as well.

### SUMMARY OF THE INVENTION

The invention provides a method for promoting cell differentiation. The method involves modifying a cell to increase expression of a BAG polypeptide that promotes differentiation of a cell, such as a neuronal cell, stem cell or neural progenitor cell.

The invention provides another method for promoting cell differentiation. The method involves modifying a cell to increase the amount of a nuclear localized BAG polypeptide, when the nuclear localized BAG polypeptide promotes differentiation of the cell.

The invention also provides method for reducing the rate of cell proliferation. The method involves modifying a cell to increase the amount of a nuclear localized BAG polypeptide, when the nuclear localized BAG polypeptide inhibits proliferation.

The invention further provides a method for suppressing apoptosis. The method involves modifying a cell to increase the amount of a nuclear localized BAG polypeptide, when the nuclear localized BAG polypeptide suppresses apoptosis.

The invention provides method for identifying the differentiation stage of a cell. The method involves (a) measuring an amount of BAG polypeptide at a subcellular location in a cell; (b) comparing the measured amount of BAG polypeptide to a reference amount of BAG polypeptide indicative of a particular differentiation stage; and (c) identifying the differentiation stage of the cell. The subcellular location can be, for example, the nucleus or cytosol. The method can be practiced by performing the additional step of measuring an amount of BAG polypeptide at a second subcellular location of a cell.

The invention also provides a method for identifying an agent that alters cell differentiation. The method involves (a) measuring an amount of BAG polypeptide at a subcellular location in a cell in the presence and absence of a candidate agent, and (b) identifying an agent that alters the amount of BAG polypeptide at the subcellular location, the agent being an agent that alters cell differentiation. In one embodiment, the method can be practiced by identifying an agent that modulates the amount of BAG polypeptide in a cell nucleus. In another embodiment, the method can be practiced by identifying an agent that modulates the amount of BAG polypeptide in a cell cytoplasm.

### BRIEF DESCRIPTION OF THE DRAWINGS

FIG. 1 shows expression of Bag1 in cultured cells.

FIG. 2 shows that Bag1 exerts a protective effect on serum-deprived cells.

FIG. 3 shows that Bag1 induces neuronal differentiation in vitro.

FIG. 4 shows that Bag1 over-expression activates the MAP kinase pathway.

FIG. 5 shows expression of Bag1 in the developing nervous system of the mouse.

FIG. 6 shows amino acid sequences for human and mouse BAG polypeptides.

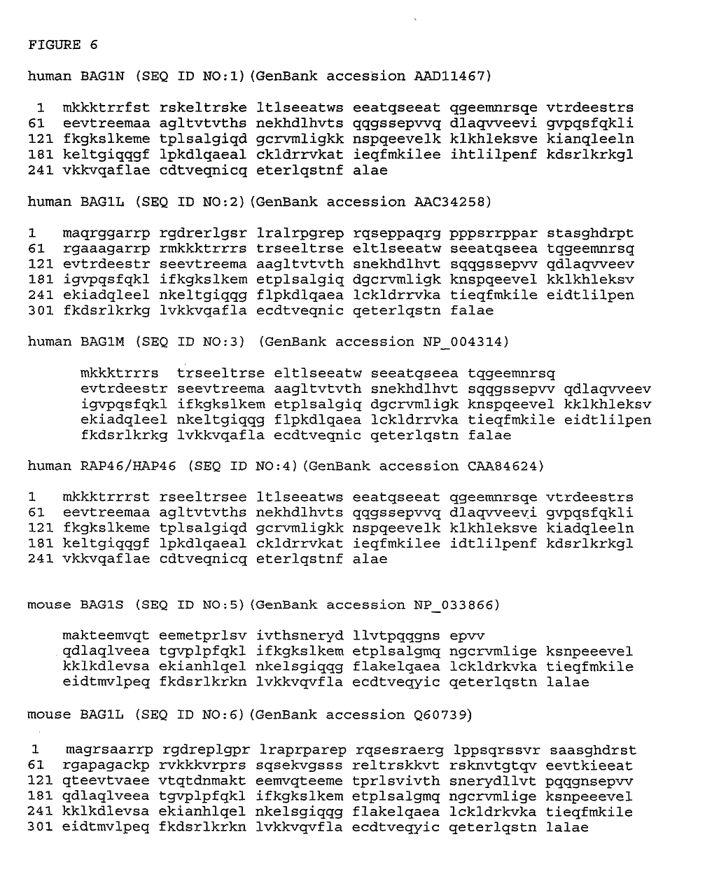

### DETAILED DESCRIPTION OF THE INVENTION

The present invention relates to the finding that modulating BAG-l expression effectively and efficiently increases differentiation of neuronal cells, including increased neuronal dendricity. Increasing dendricity leads to increased neuronal communication, thereby increasing neuronal function and performance. Thus, the present invention is useful for treating diseases or disorders marked by reduction of neuronal dendricity and function, including but not limited to Parkinson's disease, amyotrophic lateral sclerosis, Alzheimer's disease, or any other neurodegenerative disease, or physical, chemical or toxic damage to brain, spinal or peripheral nerve cells. Further, the present invention is useful for restoring or optimizing neuronal communication, function or performance.

Further, the invention provides methods for inducing pluripotent cells, such as stem cells and neuronal precursor cells, to differentiate into neurons. Such differentiated neurons can provide a means for treatment of neurodegenerative diseases and neural damage due to trauma. The methods of the invention for promoting cell differentiation can be applied to in vitro and ex vivo methods for preparing cells useful in cell-based therapies for neurological disorders and injuries. For example, cells, such as stem cells and neuronal precursors, can be taken from a patient, grown in vitro, induced to differentiate into specific neuronal cell types by increasing the amount of a nuclear localized BAG polypeptide in the cells, and re-implanted into the patient to replace lost neurons as a treatment for a variety of neurological disorders and injuries.

In addition, induction of neuronal differentiation reverses neuronal proliferative disorders. Thus, the present invention is useful for treating neuronal proliferative disorders, including cancers, and disorders in any other cell type that might be similarly affected. The methods of the invention for reducing the rate of cell proliferation can be applied to treating an individual having a neuronal cell proliferative disorder by modulating the amount of a nuclear localized BAG polypeptide in the cells of the individual. Proliferative disorders include those diseases or abnormal conditions that result in unwanted or abnormal cell growth, viability or proliferation. For example, cell proliferative disorders include diseases associated with the overgrowth of connective tissues, such as various fibrotic diseases, including scleroderma, arthritis, alcoholic liver cirrhosis, keloid, and hypertropic scarring; vascular proliferative disorders, such as atherosclerosis; benign tumors, and the abnormal proliferation of cells mediating autoimmune disease. Proliferative disorders of the central nervous system include, for example, cerebellar astrocytomas and medulloblastomas, ependymomas, gliomas, germinomas, and neuroblastoma.

The methods of the invention for suppressing apoptosis can be used in methods to extend the life of cells. It can be desired to extend the life of cells for several reasons. For example, the death of cells in tissues and organs being prepared for transport and transplant can be inhibited by modulating the amount of a nuclear localized BAG polypeptide in the cells. It is desirable to inhibit apoptosis in such tissues to prevent loss of viability of the tissues and organs. In addition, cell lines can be established for long term culture by modulating the amount of a nuclear localized BAG polypeptide in the cells. Further, several pathological conditions characterized by premature and unwanted cellular apoptosis, for example, accelerated aging disorders and neurodegenerative diseases, can be treated by increasing the amount of a nuclear localized BAG polypeptide in cells of an individual.

Further, a cell can be modified to contain a decreased amount of BAG in its nucleus in order to promote apoptosis. There are several circumstances in which it is desirable to promote apoptosis. For example, as cancer cells progress towards more aggressive forms, they often become highly resistant to drug- or radiation-induced apoptosis, generally through the loss of function p53, a gene which can trigger apoptosis in response to DNA damage. Thus, modulating the amount of BAG polypeptide in the nucleus of a cell can be used to induce apoptosis in tumor cells, and in particular, neuronal tumor cells.

The invention also provides a method for screening to identify agents that alter cell differentiation, such as those capable of inducing neural differentiation. A differentiation-inducing agent can be used to generate neurons for therapeutic purposes, or to control a disease or disorder characterized by reduced or insufficient cell differentiation.

As demonstrated herein, Bag1 expression and cellular localization play an important role in neuronal differentiation, cell proliferation and apoptosis. In particular, stable overexpression of mouse Bag1 p29 in neuronal cell line CSM14.1 promotes neuronal differentiation in these cells. During differentiation, Bag1 protein levels in CSM14.1 cells decreased in the nucleus and increased in the cytosol. Examination of Bag1 expression in vivo at various stages of mouse neuronal development revealed patterns of Bag1 protein expression and intracellular location that correlated with in vitro observations in CSM14.1 cells.

As used herein, the term “BAG polypeptide” is intended to mean a polypeptide having structural similarity to a BAG domain described in Takayama and Reed, Nature Cell Biol. 3:237-241 (2001) and capable of binding to and regulating the activity of the ATPase domain of an Hsp70 polypeptide. The term can include a human BAG polypeptide including, for example, BAG1, BAG2, BAG3, BAG4, BAG5 or BAG6. Also included in the term are isoforms of human BAG polypeptides including, for example the BAGlN, BAGlM, RAP46/HAP46, BAG1S and BAGlL isoforms of BAGl; the CAIR-1 and Bis isoforms of BAG3; the SODD isoform of BAG4 and the Scythe and BAT3 isoforms of BAG6. The term can further include BAG polypeptides from other organisms including, for example, mouse BAGl, S. cerevisiae BAGl, S. pombe BAGlA and BAGlB, C. elegans BAG1 and BAG2, Drosophila malanogaster BAG, Xenopus laevis BAG and Arabadopsis thaliana BAG1 (see, for example Takayama et al., supra (2001)).

A BAG polypeptide useful in a method of the invention can be, for example, a mammalian BAG1 polypeptide or an active modification thereof, such as mouse or human BAG1. Thus, a BAG polypeptide can have substantially the amino acid sequence of a human BAG1N (SEQ ID NO:1)(GenBank accession AAD11467), human BAG1L (SEQ ID NO:2)(GenBank accession AAC34258), human BAG1M (SEQ ID NO:3)(GenBank accession NP_{—}004314), RAP46/HAP46 (SEQ ID NO:4)(GenBank accession CAA84624), mouse BAG1N (SEQ ID NO:5)(GenBank accession NP_{—}033866) or mouse BAG1L (SEQ ID NO:6)(GenBank accession Q60739). Other GenBank accession numbers contain identical and substantially similar polypeptide sequences for each of these BAG polypeptides, each of which can be used in a method of the invention. The GenBank database also contains accession numbers for BAG nucleic acid sequences encoding BAG polypeptides. Nucleic acid sequences encoding BAG polypeptides include, for example U46917 (human BAG1N), AF022224 (human BAG1L), NM_{—}004323 (human BAG1M), Z35491 (human RAP46/HAP46), NM_{—}009736 (mouse BAG1N) and AF022223 (mouse BAG1L). Other GenBank entries contain identical or substantially similar nucleic acid sequences that encode BAG polypeptides useful in the methods of the invention. The term is intended to include polypeptides having a minor modification so long as the modification does not destroy the ability of the BAG polypeptide to carry out a specific function associated with the unmodified polypeptide.

A modified BAG polypeptide can have one or more additions, deletions, or substitutions of natural or non-natural amino acids relative to the native polypeptide sequence. A modification to a polypeptide sequence can be, for example, a conservative change, wherein a substituted amino acid has similar structural or chemical properties, for example, substitution of an apolar amino acid with another apolar amino acid (such as replacement of leucine with isoleucine). A modification can also be a nonconservative change, wherein a substituted amino acid has different but sufficiently similar structural or chemical properties so as to not adversely affect the desired biological activity, for example, replacement of an amino acid with an uncharged polar R group with an amino acid with an apolar R group (such as replacement of glycine with tryptophan). Further, a minor modification can be the substitution of an L-configuration amino acid with the corresponding D-configuration amino acid with a non-natural amino acid.

In addition, a minor modification can be a chemical or enzymatic modification to a BAG polypeptide, such as replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation.

Those skilled in the art can determine whether minor modifications to the native BAG polypeptide sequence are advantageous. Such modifications can be made, for example, to enhance the stability, bioavailability or bioactivity of the BAG polypeptide. A modified BAG polypeptide can be prepared, for example, by recombinant methods, by synthetic methods, by post-synthesis chemical or enzymatic methods, or by a combination of these methods, and tested for a specific function associated with the unmodified BAG polypeptide.

It is understood that minor modifications of primary amino acid sequence can result in polypeptides which have substantially equivalent or enhanced function as compared to a reference BAG polypeptide sequence. These modifications can be deliberate, as through site-directed mutagenesis, or can be accidental such as through mutation in hosts harboring an encoding nucleic acid. All such modified polypeptides are included in the definition of a BAG polypeptide so long as the ability of a BAG polypeptide to modulate cell differentiation, proliferation or apoptosis is retained. Further, various molecules can be attached to a BAG polypeptide, for example, other polypeptides, such as cell-localization targeting sequences, carbohydrates, lipids, or chemical moieties. Such modifications are included within the definition of each of the polypeptides of the invention.

Those skilled in the art also can determine regions in a BAG amino acid sequence that can be modified without abolishing BAG polypeptide activity. Structural and sequence information can be used to determine the amino acid residues important for BAG polypeptide activity. For example, comparisons of amino acid sequences of BAG polypeptide sequences from different species can provide guidance in determining amino acid residues that can be altered without abolishing activity. A comparison of BAG domain amino acid sequences of BAG polypeptides from human, yeast and nematode is shown, for example, in Takayama, 2001, supra. In addition, computer programs known in the art can be used to determine which amino acid residues of a BAG polypeptide, such as a mammalian BAG polypeptide, can be modified as described above without abolishing activity (see, for example, Eroshkin et al., Comput. Appl. Biosci. 9:491-497 (1993)).

BAG polypeptides included in the term can contain one or more of a diversity of domains which allow them to bind with specific target polypeptides or which target them to specific cellular locations. BAG1 and BAG6, for example, contain a ubiquitin-like domain which can bind to the 26S proteosome. BAGlL contains a nuclear localization signal capable of directing the polypeptide to the cell nucleus. BAGlL and BAGlM contain eight copies of a TXSEEX repeat that mediates binding to DNA. Further functions of BAG1 include, the ability to bind specifically to Bcl2 in an ATP-hydrolysis-dependent manner, the ability to bind to the serine/threonine kinase Rafl to stimulate activity of the kinase, the ability to bind to and inhibit Siahl, the ability to bind to receptors such as the human growth factor receptor (HGFR), platelet derived growth factor receptor (PDGFR), steroid receptors and retinoic acid receptor (RAR). BAG3 contains a WW domain and is capable of binding specifically to polypeptides in a phosphorylation-dependent manner as described, for example, in Lu et al. Science 283:1325-1328 (1999). BAG3 can bind to SH3 containing polypeptides including, for example, phospholipase C-γ and contains several PXXP motifs for binding to SH3 domains. BAG3 can further bind to Bcl2 and epidermal growth factor. BAG4 binds to the death domains of tumor necrosis factor receptor 1 and death receptor 3, preventing cell death signaling and NF-kB induction by suppressing ligand independent receptor oligomerization. BAG6 is nuclear localized and is capable of binding to the apoptosis inducing polypeptide, Reaper.

As used herein the term “proliferate,” when used in reference to a cell, is intended to refer to the process or result of a cell dividing to yield two viable cells. The term can include proliferation that occurs normally in the growth, development or maintenance of a population of cells, tissue or organism. The term can also include abnormal proliferation associated with a disease or condition such as cancer, autoimmune disease, trauma, Alzheimer's disease, AIDS, stroke, Huntington's disease, Parkinson's disease, amyotrophic lateral sclerosis, systemic lupus erythematosis, multiple sclerosis, diabetes mellitus or rheumatoid arthritis.

As used herein the term “differentiate,” when used in reference to a cell, is intended to refer to the process or result of a cell changing from a precursor cell to a specialized cell. The term can include changes characterized by losing one or more identifiable characteristics of a precursor cell or acquiring one or more characteristics of a specialized cell. The term includes changes associated with normal development of a cell including, for example, changes to a subsequent differentiation stage occurring for a neural cell as it passes from El to maturity.

As used herein, the term “inhibit,” when used in reference to a biological activity, is intended to mean reducing the rate at which the biological activity occurs or the magnitude of the effect of the biological activity. The term can include a transitory or permanent reduction of the biological activity and can be, for example, a partial or total loss of the activity.

As used herein, the term “activate,” when used in reference to a biological activity, is intended to mean increasing the rate at which the biological activity occurs or the magnitude of the effect of the biological activity. The term can include a transitory or permanent increase of the biological activity and can be, for example, an initiation of the activity.

As used herein, the term “subcellular location” is intended to mean a surface or internal volume of an organelle or cell component. The term can include, for example, the nucleus, cytosol, mitochondria, membrane, endoplasmic reticulum, golgi apparatus or ribosome. The term “cytosol” is intended to mean the aqueous phase of the cell cytoplasm excluding the surface or internal volume of organelles such as the nucleus or mitochondria.

As used herein, the term “modifying,” when used in reference to a cell, is intended to mean changing the structure or activity of the cell. The term includes, for example, changing the cell by adding an agent or stimulus to the cell, deleting a naturally occurring component of the cell or increasing or decreasing the amount of a naturally occurring component of the cell. The change can be permanent or transient so long as it is sufficient to alter a structure or activity of the cell. Structures that can be altered include, for example, nucleic acids, polypeptides, metabolites, signaling molecules, or organelles. Functions that can be altered include, for example, translation or transcription of a nucleic acid; catalytic activity of a polypeptide; post-translational modification of a polypeptide; stability of a polypeptide, nucleic acid, metabolite or signaling molecule; or cell proliferation or differentiation. The term can include, for example, adding a recombinant nucleic acid molecule that expresses a polypeptide of interest, an antisense nucleic acid molecule that inhibits expression of a polypeptide of interest, or a molecule that induces or represses transcription or translation from a nucleic acid.

As used herein, the term “stem cell” is intended to mean a pluripotent cell type which can differentiate under the appropriate conditions to give rise to all cellular lineages. Thus, a stem cell differentiates to neuronal cells, hematopoietic cells, muscle cells, adipose cells, germ cells and all other cellular lineages. A stem cell can be an embryonic stem cell.

As used herein, the term “progenitor cell” means any cell capable of differentiating into the desired cell type, such as a neuronal cell, under the appropriate conditions. Progenitor cells can be multipotent or unipotent and can be stem cells, precursor cells, primary cells or established cells. Progenitor cells such as stem cells generally are distinct from neurons in that they lack neuronal markers such as the nuclear protein NeuN, neurofilament and microtubule-associated protein 2 (MAP2) as well as the neuronal-like processes characteristic of mature neurons.

As used herein, the term “reducing the rate of cell proliferation” is intended to mean slowing or decreasing the number of cell divisions per unit time. Such a decrease in cell proliferation can restore more normal proliferative characteristics on an abnormally proliferating cell.

Bag1 (Bcl-2-associated athanogene-1) was the first identified member of a family of Hsp70-binding proteins containing a conserved C-terminal region termed the “Bag domain” (Takayama et al., Cell 80:279-284 (1995); Takayama et al., J. Biol. Chem. 274:781-786 (1999); Takayama and Reed, Nature Cell Biol., in press (2001)). The Bag domain binds tightly to the ATPase domain of the Hsp70 family of molecular chaperones and regulates their activity. Diversity in the N-terminal regions of BAG-family proteins permits their association with specific target proteins or targeting to subcellular locations.

Bag1 was identified by virtue of its ability to bind and collaborate with Bcl-2 in suppressing cell death (Takayama et al., supra). Since then, multiple functions have been reported for Bagl, including interactions with the serine/threonine-specific protein kinase Raf, some tyrosine kinase growth factor receptors, and several steroid hormone receptors (Zeiner and Gehring, Proc. Natl. Acad. Sci. USA 92:11465-11469 (1995); Bardelli et al., EMBO J. 15:6205-6212 (1996); Wang et al., Proc. Natl. Acad. Sci. USA 93:7063-7068 (1996)). At the cellular level, over-expression of Bag1 can result in various phenotypes, including enhanced tumor cell proliferation, promotion of cell motility and metastasis, and increased resistance to apoptosis (Takayama et al., Cell 80:279-284 (1995); Takayama et al., J. Biol. Chem. 274:781-786 (1999); Bardelli et al., EMBO J. 15:6205-6212 (1996); Clevenger et al., Mol. Endocrinol. 11:608-618 (1997); Danen-van Oorschott et al., Apoptosis 2:395-402 (1997); Schultz et al., J. Neurochem. 69:2075-2086 (1997); Takaoka et al., Oncogene 14:2971-2977 (1997); Liu et al., J. Biol. Chem. 273:16985-16992 (1998); Kullmann et al., J. Biol. Chem. 273:14620-14625 (1998); Matsuzawa et al., EMBO J. 17:2736-2747; Froesch et al., J. Biol. Chem. 273:11660-11666 (1998)).

Previous studies have shown that in both mouse and humans, there is only a single size of BAG-l mRNA molecules transcribed, but a plurality of sizes of BAG-l polypeptides are translated (Takayama et al., Cancer Research 58:3116-3131 (1998)). In mouse, two isoforms have been identified: the normal length BAG-1 (referred to herein as “BAG-l N”); and a longer BAG-1 polypeptide, or BAG-1L. In human, in addition to the presence of the corresponding BAG-1N and BAG-1L isoforms, a medium length BAG-l polypeptide, referred to as BAG-lM, is also translated. The cDNA complementary to the BAG-l mRNA for mouse and human are set forth in U.S. Pat. No. 5,539,094 and in Takayama et al., supra, (each of which are incorporated herein by reference in their entirety).

The present invention relates to the observations that increasing the amount of BAG polypeptide in a cell, and particularly in a cell nucleus, modulates cell differentiation, growth and apoptosis.

Therefore, the invention provides a method for promoting cell differentiation. In one embodiment, the method involves modifying a cell to increase expression of a BAG polypeptide that promotes differentiation of the cell. In another embodiment, the method involves modifying a cell to increase the amount of a nuclear localized BAG polypeptide that promotes differentiation of the cell.

The biochemical pathways of cell differentiation, proliferation and apoptosis appear to be mutually exclusive, such that proliferation and apoptosis are inhibited in a cell undergoing differentiation. Therefore, the methods of the invention for promoting differentiation can be used to reduce the rate of cell proliferation. Accordingly, the invention provides a method for reducing the rate of cell proliferation. The method involves modifying a cell to increase the amount of a nuclear localized BAG polypeptide, when the BAG polypeptide inhibits proliferation.

The invention also provides a method for suppressing apoptosis. The method involves modifying a cell to increase the amount of a nuclear localized BAG polypeptide, when the BAG polypeptide suppresses apoptosis. Suppressing apoptosis refers to reducing or inhibiting the process of programmed cell death. Programmed cell death is a regulated process in which a cell responds to a specific physiological or developmental signal and undergoes a programmed series of events that leads its death and removal from the organism. Examples of the cellular events that characterize apoptosis are cell shrinkage, mitochondrial break down with the release of cytochrome c, cell surface blebbing, chromatin degradation, and phosphotidylserine exposure on the surface of the plasma membrane. Apoptosis is distinct from necrosis, or cell death that results from injury, which is characterized by overall cell and organelle swelling, with subsequent early loss of membrane integrity followed by cell and organelle lysis. Necrosis, unlike apoptosis, is accompanied by an inflammatory response in vivo.

The methods of the invention for promoting cell differentiation can be applied to a variety of cell types, including non-neuronal and neuronal progenitor cells and other neuronal cell types. In one embodiment, the cell to be differentiated is a stem cell. Stem cells and neuronal precursor cells provide a renewable source of replacement cells and tissues for treating a variety of neurological injuries and diseases, including spinal cord injury, Parkinson's and Alzheimer's diseases and cancer. Stem cells include pluripotent cells derived from an adult or embryo. An embryonic stem cell, or ES cell, is a pluripotent cell type derived from an embryo which can differentiate to give rise to all cellular lineages. Examples of cell markers that indicate a human embryonic stem cell include the Oct-4 transcription factor, alkaline phosphatase, SSEA-4, TRA1-60, and GCTM-2 epitope as described in Reubinoff et al., supra, 2000.

In another embodiment, the cell to be differentiated is a progenitor cell. A progenitor cell useful in the invention can be multipotent or unipotent. A multipotent or pluripotent progenitor cell is capable of differentiating into two or more distinct lineages, including the neuronal lineage. Multipotent progenitor cells such as stem cells, which are generally nestin-positive cells, are distinguished from unipotent precursor cells, which are generally Hu-positive cells. Expression of nestin and Hu can be determined, for example, by immunochemical methods. A multipotent progenitor cell is capable of differentiating into at least three or more, four or more, or five or more distinct lineages, including the neuronal lineage.

In a further embodiment, the cell to be differentiated is a neuronal cell. A neuronal cell having an immature or not fully differentiated phenotype can be differentiated to a more mature or complete differentiated state. A differentiated state of a neuronal cell can be indicated phenotypically, for example, by an increased number of extended neurites, increased length of extended neurites, decreased cell body size, and increased expression of neuronal markers, as compared to a non-differentiated state, as well as functionally.

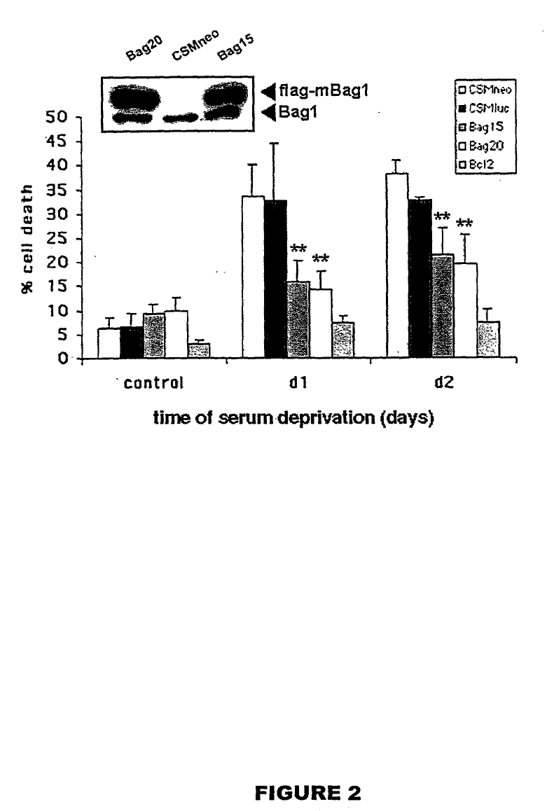

The methods of the invention are useful for differentiating a progenitor cell, including a stem cell and neuronal progenitor cell, to produce a neuronal cell. Neuronal cells are nerve cells characterized, in part, by containing one or more markers of neuronal differentiation. Examples of cell markers that indicate a differentiated neuronal cell including neurofilament proteins, β-tubulin, Map2a+b, synaptophysin, glutamic acid decarboxylase, TuJ1, SNAP 25, transcription factor Brn-3, and GABA_{A} α2 receptor subunit as described in Reubinoff et al., Nat. Biotech. 18:399-404 (2000); Ghosh and Greenberg, Neuron 15:89-103 (1995); Bain et al., Devel. Biol. 168:342-357 (1995); and Williams et al., Neuron 18:553-562 (1997). A neuronal cell further generally is characterized as containing neuronal-like processes, or neurites, as shown in FIG. 2B. A neuronal phenotype further can be characterized by cellular changes such as reduced cell body size, an increase in the number of neurites and an increase in the length of neurites.

The amount of BAG in a cell can be modulated in vitro, ex vivo, in situ or in vivo, for example, by increasing expression of BAG from an exogenous nucleic acid molecule, by introducing a BAG polypeptide or functional analog thereof into a cell, by modulating the expression or activity of a gene or protein product that regulates BAG amount or localization in a cell. In one embodiment, a recombinant nucleic acid molecule containing a nucleotide sequence encoding a BAG polypeptide capable of modulating cell differentiation, cell proliferation or apoptosis, and operatively linked to a promoter of gene expression, is introduced into a cell.

A variety of methods are known in the art for introducing a nucleic acid molecule into a cell, including a progenitor cell, stem cell or neuronal cell. Such methods include microinjection, electroporation, lipofection, calcium-phosphate mediated transfection, DEAE-Dextran-mediated transfection, polybrene- or polylysine-mediated transfection, and conjugation to an antibody, gramacidin S, artificial viral envelopes or other intracellular carriers such as TAT. For example, cells can be transformed by microinjection as described in Cibelli et al., Nat. Biotech. 16:642-646 (1998) or Lamb and Gearhart, Cur. Opin. Gen. Dev. 5:342-348 (1995); by lipofection as described in Choi (U.S. Pat. No. 6,069,010) or Lamb and Gearhart, Cur. Opin. Gen. Dev. 5:342-348 (1995); by electroporation as described in Current Protocols in Molecular Biology, John Wiley and Sons, pp 9.16.4-9.16.11 (2000) or Cibelli et al., Nat. Biotech. 16:642-646 (1998); or by fusion with yeast spheroplasts Lamb and Gearhart, Cur. Opin. Gen. Dev. 5:342-348 (1995).

A nucleic acid encoding a BAG polypeptide can be delivered into a mammalian cell, either in vivo or in vitro using suitable vectors well-known in the art. Suitable vectors for delivering a nucleic acid encoding a BAG polypeptide to a mammalian cell, include viral vectors such as retroviral vectors, adenovirus, adeno-associated virus, lentivirus, herpesvirus, as well as non-viral vectors such as plasmid vectors. Such vectors are useful for providing therapeutic amounts of a BAG polypeptide.

Viral based systems provide the advantage of being able to introduce relatively high levels of the heterologous nucleic acid into a variety of cells. Suitable viral vectors for introducing an invention nucleic acid encoding a BAG polypeptide into a mammalian cell are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et_al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)). It is understood that both permanent and transient expression can be useful in a method of the invention.

A BAG polypeptide-encoding recombinant nucleic acid can be directed into a particular tissue or organ system, for example, by vector targeting or tissue-restricted gene expression. Therefore, a vector useful for therapeutic administration of a nucleic acid encoding an a BAG polypeptide can contain a regulatory element that provides tissue specific expression of the BAG polypeptide. For example, a nucleic acid sequence encoding a BAG polypeptide can be operatively linked to a neuronal cell specific promoter.

Any of a variety of inducible promoters or enhancers can also be included in a nucleic acid or vector of the invention to allow control of expression of a BAG polypeptide by added stimuli or molecules. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.); metallothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse ammary tumor virus (MMTV) induced by steroids such as glucocorticoid and estrogen (Lee et al., Nature, 294:228-232 (1981); and heat shock promoters inducible by temperature changes.

An inducing agent can be added to a cell to increase expression of a nucleic acid molecule encoding a BAG polypeptide. An inducing agent can be a substance or condition, such as a chemical, peptide, polypeptide, or wavelength of radiation, that triggers transcription of a nucleic acid sequence encoding a BAG polypeptide. An inducing agent can trigger transcription through a variety of different mechanisms, including for example, release of a repressor of transcription, activation of a promoter of transcription, and activation of a component of transcription machinery. An inducible system particularly useful for therapeutic administration utilizes an inducible promoter that can be regulated to deliver a level of therapeutic product in response to a given level of drug administered to an individual and to have little or no expression of the therapeutic product in the absence of the drug. One such system utilizes a Gal4 fusion that is inducible by an antiprogestin such as mifepristone in a modified adenovirus vector (Burien et al., Proc. Natl. Acad. Sci. USA, 96:355-360 (1999). The GENE SWITCH inducible expression system (U.S. Pat. Nos. 5,935,934 and 5,874,534) is an example of such a system. Other inducible systems use the drug rapamycin to induce reconstitution of a transcriptional activator containing rapamycin binding domains of FKBP12 and FRAP in an adeno-associated virus vector (Ye et al., Science, 283:88-91 (1999)), use tetracycline to control transcription (Baron Nucleic Acids Res. 25:2723-2729 (1997)) and use synthetic dimerizers to regulate gene expression (Pollock et al., Methods Enzymol. 306:263-281 (1999)). Such a regulatable inducible system is advantageous because the level of expression of the therapeutic product can be controlled by the amount of drug administered to the individual or, if desired, expression of the therapeutic product can be terminated by stopping administration of the drug.

A BAG polypeptide can be delivered into a cell, including specific delivery into the nucleus or cytoplasm of a cell using a variety of drug delivery methods. One method for delivering a BAG polypeptide into a cell nucleus is to fuse the BAG polypeptide to a targeting sequence, such as a well-known nuclear targeting sequence. For example, a BAG polypeptide can be fused at the amino or carboxyl terminus with a nuclear-targeting sequence of antennapedia or VP22. A BAG polypeptide also can be delivered to a cell as a TAT/MEF2 polypeptide fusion by techniques well known in the art as described in Nagahara et al., Nature Medicine 4:1449-1452 (1998). Such translocating peptide sequences can be used to deliver a BAG polypeptide into a cell in culture or within an individual (see, for example, Aints et al., J Gene Med 1:275-279, (1999) and Dilber et al., Gene Therapy 6: 12-21, (1999)). A cytoplasmic targeting sequence, including a nucleus-excluding sequence, also can be fused to a BAG polypeptide for use in the methods of the invention for modulating cell differentiation, proliferation and apoptosis. Whether a nuclear-targeting sequence or cytoplasmic-targeting sequence is used will depend on the application of the method. For example, when it is desired to promote differentiation, reduce cell proliferation or suppress apoptosis, a BAG polypeptide capable of promoting differentiation, reducing cell proliferation or suppressing apoptosis can be fused to a nuclear-targeting sequence.

A BAG polypeptide, including a BAG polypeptide containing a nuclear- or cytoplasmic-targeting sequence, can be delivered into a cell using a variety of drug delivery systems designed for use with polypeptides, including nasal delivery systems, osmotic-based approaches, liposomal systems and bioerodible polymers, nanoparticles, microparticles, and membranes for targeted and regulated polypeptide delivery.

The methods of the invention for modulating cell differentiation, proliferation and apoptosis involve determining the amount of BAG polypeptide in a cell, either prior to modifying a cell to alter BAG polypeptide amount, after such modification, or both. BAG polypeptide levels can be determined by detection of a BAG polypeptide or mRNA encoding a BAG polypeptide, or both. A BAG polypeptide level is intended to mean the amount, accumulation or rate of synthesis of a biochemical form of a BAG polypeptide in a cell. The polypeptide level can be represented by, for example, the amount or rate of synthesis of the polypeptide, a precursor form or a post-translationally modified form of the polypeptide. Various biochemical forms of a polypeptide resulting from post-synthetic modifications can be present in a biochemical system. Such modifications include post-translational modifications, proteolysis, and formation of macromolecular complexes. Post-translational modifications of polypeptides include, for example, phosphorylation, lipidation, prenylation, sulfation, hydroxylation, acetylation, addition of carbohydrate, addition of prosthetic groups or cofactors, formation of disulfide bonds and the like. Accumulation or synthesis rate with or without such modifications is included with in the meaning of the term. Similarly, a BAG polypeptide level also refers to an absolute amount or a synthesis rate of the polypeptide determined, for example, under steady-state or non-steady-state conditions.

A variety of well-known immunological and nucleic acid techniques can be used to determine if a BAG polypeptide or mRNA encoding a BAG polypeptide is present in a cell, including whether the BAG polypeptide is present in the nucleus or cytoplasm of the cell. Such methods can be used to monitor BAG polypeptide levels either directly or indirectly. Exemplary methods include western blotting, two-dimensional gels, methods based on protein or peptide chromatographic separation, methods that use protein-fusion reporter constructs and colorimetric readouts, methods based on characterization of actively translated polysomal mRNA, and mass spectrometric detection. Additionally, aptamers can be used to detect specific polypeptides in a sample. Aptamers are oligonucleotides having binding affinity for polypeptides (Tuerk and Gold, Science 249:505-510 (1990); Ellington and Szostak, Nature 346:818-822 (1990); Joyce, Curr. Opin. Struct. Biol. 4:331-336 (1994); Gold et al., Annu. Rev. Biochem. 64:763-797 (1995); Jayasena, Clin. Chem. 45:1628-1650 (1999); Famulok and Mayer, Curr. Top. Microbiol. Immunol. 243:123-136 (1999)).

The amount of BAG polypeptide in a cell can be detected by measuring levels of BAG protein using agents that bind specifically to a BAG polypeptide. Such agents can be labeled for detection using methods well known to those of skilled in the art. A variety of agents can be used to specifically detect BAG protein, including proteins known to bind specifically to BAG, antibodies to BAG (such as described in U.S. Pat. No. 5,641,866, incorporated herein by reference in its entirety), or peptides which specifically bind BAG.

Other, non-antibody proteins, may also be used as “agents.” For example, BAG proteins are known to specifically bind numerous proteins, such as Bcl-1, Bcl-2, Raf-1, HGF-receptor, PDGF-receptor, Hsp70, Hsc70, steroid hormone receptors, and the like. As a result, any of these proteins, or active BAG binding fragments thereof, may be used to specifically bind BAG. An exemplary active binding fragment of a protein which binds BAG-1 (and also BAG-2 and BAG-3) is a BAG binding domain of Hsp70. The ATPase domain of Hsp70 may be expressed in a truncated form, lacking the carboxyl-terminal peptide-binding domain. In this form, Hsp70 will not indiscriminately bind proteins in non-native conformations; however, the ATPase domain of Hsp70 is still capable of binding BAG-1 (or BAG-2 or BAG-3) protein. Therefore, an actively binding fragment of a protein known to specifically bind BAG may be used as an “agent” which specifically binds BAG protein.

Antibodies, both monoclonal and polyclonal, may be used as specifically binding agents which bind BAG protein or a polypeptide fragment thereof. Also contemplated herein as BAG binding agents are any mutants of proteins which specifically bind BAG, whether by deletion (as above exemplified), addition (e.g., addition of a GST domain or a GFP domain), or sequence modification (for example, site-specific mutagenesis), and the like. BAG antibodies useful in the methods of the invention include those described in Example VII, which can be used for detecting BAG polypeptide using Western blotting and in situ hybridization methods. In addition, Takayama et al., supra, describes preparation of an antibody that binds to all isoforms of BAG-l.

To determine if BAG polypeptide is contained in either or both the nucleus or cytoplasm, a variety of immunocytochemical and biochemical methods can be employed. Immunocytochemical methods for detecting nuclear- or cytoplasm-localized BAG polypeptide can involve detection of a BAG binding agent bound to a BAG polypeptide in a living or fixed cell, including a cell within a tissue, using microscopy. A variety of nuclei-specific stains, such as DAPI, for example, can be used to confirm nuclear localization of a BAG polypeptide. Biochemical methods for detecting nuclear- or cytoplasm-localized BAG polypeptide can involve separating a nuclear and cytoplasmic cell fraction and separately detecting a BAG binding agent bound to a BAG polypeptide in each fraction. A variety of well-known cell fractionation methods can be used to separate a cell nucleus from cytoplasm. Detection of a level of BAG polypeptide can be qualitative or quantitative.

Preparation of the agent for use in the detection of BAG protein levels will be carried out using the methods of one of ordinary skill in the art, such as the methods exemplified in the Current Protocols in Molecular Biology, and in U.S. Pat. No. 5,882,864. Similarly, detection of BAG protein levels may be carried out using any of the methods known to one of ordinary skill in the art including histochemical staining, Western Blot analysis, immunoprecipitation (or the equivalent thereof for non-antibody agents), and the like. In a preferred embodiment of the invention, the method of detecting BAG protein levels is an immunoassay (such as an ELISA, immuno-PCR, and the like), which includes the use of at least one antibody. Measurement of the polypeptide encoded by a BAG gene may include measurements of fragments of the polypeptide, wherein the fragments arise from transcriptional or translational variants of the gene; or alternatively, differently sized polypeptides arise as a result of post translational modifications including proteolysis of a larger portion of a BAG polypeptide.

An exemplary immunoassay for use in the invention methods for detecting BAG protein levels is an immuno-polymerase chain reaction immuno-PCR assay (described in U.S. Pat. No. 5,665,539, which is incorporated herein in its entirety). Immuno-PCR utilizes an antibody (or other agent which binds BAG) to detect the BAG protein, wherein the antibody (or other agent) is linked to a molecule (typically biotin) which specifically binds a bridging molecule (typically avidin), wherein this bridging molecule is capable of binding a second molecule (typically biotin) attached to a nucleic acid marker. This nucleic acid marker is then amplified using PCR methods. This sensitive detection method is particularly useful when BAG levels are often difficult to detect by other methods, for example, detection of BAG in serum.

The methods of the invention can be applied to small samples such as cells removed from a particular tissue or tumor. Methods well known in the art for amplification of mRNA, such as, for example, PCR-based amplification and template-directed in vitro transcription (IVT) can be used for generating a sample to be used in the methods of the invention. Methods of amplifying nucleic acids by reverse transcription are well known to those skilled in the art (see, for example, Dieffenbach and Dveksler, PCR Primer: A Laboratory Manual, Cold Spring Harbor Press (1995)).

Measurement of the polypeptide encoded by a BAG gene may further be carried out to specifically measure: (a) the level of BAG produced in the entire cell, (b) the level of BAG produced in the cytosol, (c) the level of BAG produced in the nucleus, (d) level of BAG present in cell-free extract, and (e) any combination thereof. Exemplary methods which can be used in such measurements include methods such as histochemical staining, particularly differential staining between the cytosol and the nucleus, Western blot analysis of nuclear extracts, cytosolic extracts, or serum.

Detection of levels of mRNA encoding BAG may also serve as an indicator of BAG expression. The methods used to detect mRNA levels will include the detection of hybridization or amplification with the mRNA encoding BAG. This detection may be carried out by analysis of mRNA either in vitro or in situ (e.g., in a tissue sample) using one of the methods known to one of ordinary skill in the art as exemplified in the Current Protocols in Molecular Biology (John Wiley & Sons, 1999); in U.S. Pat. No. 5,882,864; and the like. A BAG mRNA detected will be any RNA transcript of a BAG gene, or fragment thereof.

Detection of the DNA encoding BAG may also be used as an indicator of BAG expression. A plurality of changes from wild type in the portion of DNA which constitutes a BAG gene may influence levels of gene expression. For example, gene amplification will provide more copies of a BAG gene within each cell, thereby facilitating the manufacture of an increased number of mRNA molecules encoding BAG, which may result in an increased level of BAG protein within the cell. In another example, gene translocation or partial gene deletion may have an effect on the rate of gene expression by, for example, decreasing the ability of a repressor protein to repress BAG transcription, resulting in higher levels of BAG protein within the cell.

The amount of BAG polypeptide contained within a cell nucleus can be compared to the amount of BAG polypeptide contained within a cell cytoplasm using a variety of comparative methods. Such comparative methods can involve qualitative observations or quantitative measurements of BAG polypeptide amount. The amount of BAG polypeptide in a cellular localization can be determined as an absolute amount, a relative amount, or as a ratio.

The methods of the invention for modulating the amount of BAG polypeptide in a cell to induce cell differentiation, reduce the rate of proliferation of induce apoptosis can be practiced on cells in vitro, ex vivo, in situ or in vivo. As such, the methods and products of the methods can be used to treat neurodegenerative disorders, neuropathies, and proliferative disorders by providing cells in vitro or ex vivo differentiated cells to an individual, or by inducing differentiation of cells within the individual. Exemplary neurodegenerative disorders and neuropathies include diffuse cerebral cortical atrophy, Lewy-body dementia, Pick disease, mesolimbocortical dementia, thalamic degeneration, bulbar palsy, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and ophthalmoplegia.

Methods for modulating the amount of BAG polypeptide in a cell, including methods for modulating the amount of BAG polypeptide in a particular cellular localization can be practiced in in vitro, ex vivo, in situ and in vivo settings. For in vitro applications of the methods for modulating cell differentiation, reducing the rate of cell proliferation and suppressing apoptosis, a variety of cultured cells may be used, including a variety of cell lines, such as CSM 14.1, NB41A3, NT2, NSC-34, CSM-25, B65, other characterized cell lines and newly generated cell lines, and primary cells isolated from an animal. Methods for generating neuronal cell lines are well known to those skilled in the art. In addition, the methods of the invention are applicable to a large number of neuronal cell lines described in the literature, including a variety of commercially available cell lines. The differentiation state, proliferation state and apoptotic state of such cell lines can be determined using methods well known to those skilled in the art, including those methods described herein. Those skilled in the art also will know how to select appropriate growth media and conditions for growing a selected cell type, and will know to obtain and propagate cells, including adherent cells, nonadherent cells, immortalized cells and primary cells.

For ex vivo applications of the methods for modulating cell differentiation, proliferation and apoptosis, cells are treated outside of the body. Therefore, an ex vivo cell culture method involves harvesting cells from an individual. Ex vivo culture methods are applicable to a cell harvested from any tissue or organ of an individual. Cell culture conditions of ex vivo cultures include a variety of compositions. Cells can be in a heterogeneous mixture or can be isolated cells. Medium can be an undefined or defined cell culture medium or can contain added factors, including protein factors and chemical reagents in addition to substances used to modulate BAG polypeptide amount or cellular localization. Components to be included in cell culture medium will depend on the requirements of the cell type cultured. Those skilled in the art will be able to determine an appropriate cell culture medium for a selected cell type, and such media are commercially available (for example, short-term maintenance media for neuronal cells, HIBERNATE A and HIBERNATE B are available from INVITROGEN Corporation, Carlsbad, Calif. and NCM (Neuron Culture Medium) is available from CLONEXPESS, Incorporated, Gaithersburg, Md.).

For in situ and in vivo applications of the methods for modulating cell differentiation cell proliferation and apoptosis, cells can be obtained from or be present within the body of an animal, or within a bodily fluid or tissue removed from the animal. For example, the amount of BAG polypeptide contained in a cell, or within the nucleus of the cell, can be altered within a neuron in the peripheral or central nervous system, or within a neural progenitor cell or other pluripotent cell within an animal.

Cells for use in the methods of the invention can be obtained from a mammal, such as a mouse, rat, pig, goat, monkey or human, or a non-mammal containing a cell in which the amount of BAG polypeptide contained in the cell, and in particular the cell nucleus, can modulate the differentiation, proliferation or apoptotic state of the cell.

The methods of the invention involve determining one or more of the differentiation state, proliferation state or apoptotic state of a cell. The effect of modulating the amount of BAG polypeptide in a cell can be assessed by determining the differentiation state, proliferation state or apoptotic state of a cell prior to and after modulating the amount of BAG polypeptide in a cell.

A variety of methods can be used to determine the differentiation state of a cell, for example, to determine if modifying BAG polypeptide in a cell results in altering cell differentiation. In particular, the effect modifying a cell to increase the amount or localization of BAG polypeptide can be assessed by several criteria well known in the art. For example, a neuronal phenotype generally is characterized by an increased number of extended neurites, increased length of extended neurites, decreased cell body size, and increased expression of neuronal markers, as compared to a non-neuronal phenotype. Many neuronal markers are known to those skilled in the art, and include, for example, GAD 67, dye FM 1-43, TAG-1, MAP-2, and NeuN.

A variety of methods can be used to determine the proliferation state of a cell, for example, to determine if modifying the amount of BAG polypeptide in a cell alters the rate of cell growth. In particular, the effect of modifying a neoplastic or cancer cell by modifying the cell to increase the amount or localization of BAG polypeptide can be assessed by several criteria well known in the art. For example, a neoplastic or cancer cell can be distinguished from a normal cell by the uncontrolled growth and invasive properties characteristic of cancer cells. Using histological methods, a cancer cell can be observed to invade into surrounding normal tissue, have an increased mitotic index, and increased nuclear to cytoplasmic ratio, altered deposition of extracellular matrix, and a less differentiated phenotype. The unregulated proliferation of a cancer cell can be characterized by anchorage independent cell growth, proliferation in reduced-serum medium, loss of contact inhibition, and rapid proliferation compared to normal cells. Those skilled in the art will know how to determine if modifying the cell to increase the amount of nuclear localized BAG polypeptide is effective in promoting a more normal phenotype in a cancer cell. Those skilled in the art will also be able to detect a cancer cell in a population of cells, tumor, or organ.

Animal models of hyperproliferative diseases similarly can be used to assess the effect of modifying a cell to increase the amount of nuclear localized BAG polypeptide. Animal models of such pathological conditions well known in the art which are reliable predictors of treatments in humans include, for example, animal models for tumor growth and metastasis and autoimmune disease. These models generally include the inoculation or implantation of a laboratory animal with heterologous tumor cells followed by simultaneous or subsequent administration of a therapeutic treatment. The efficacy of the treatment is determined by measuring the extent of tumor growth or metastasis. Measurement of clinical or physiological indicators can alternatively or additional be assessed as an indicator of treatment efficacy. Exemplary animal tumor models can be found described in, for example, Brugge et al. Origins of Human Cancer, Cold Spring Harbor Laboratory Press, Plain View, N.Y., (1991).

Similarly, animal models predictive for neurodegenerative diseases are known in the art and can be used to assess the efficacy of treatment by measuring appropriate experimental endpoints or clinical or physiological indicators which will depend on the particular animal model selected. Those skilled in the art will know which other animal models can be used for determining the effect of modifying a cell to increase the amount of nuclear localized BAG polypeptide.

A variety of methods can be used to determine the apoptosis state of a cell, for example, to determine if modifying the amount of BAG polypeptide in a cell affects apoptosis. In particular, the effect on apoptosis of modifying a neuronal cell by modifying the cell to increase the amount or localization of BAG polypeptide can be assessed by several criteria well known in the art. Indicators of an apoptotic state of a cell include a characteristic pattern of morphological, biochemical and molecular changes, which may be broadly and chronologically defined as morphological changes, including cell shrinkage, cell shape change, condensation of cytoplasm, condensation of chromatin, nuclear envelope changes, nuclear fragmentation, loss of cell surface structures, presence of apoptotic bodies, cell detachment, phagocytosis of remains; and functional or biochemical changes, including free calcium ion concentration rise, bcl2/BAX interaction cell dehydration, loss of mitochondrial membrane potential, proteolysis, phosphotidylserine externalization, lamin B proteolysis, DNA denaturation 50-300 kb cleavage DNA fragments produced, intranucleosomal cleavage, protein cross-linking.

The effect of modulating the amount or localization of BAG polypeptide on apoptotic state of cell can be determined, for example, by treating a cell or animal to modulate BAG polypeptide amount, expression or localization and by observing the number of apoptotic cells in a cell population, or the phenotype, of the animal, as compared to the number of apoptotic cells in a population of untreated cells or the phenotype of an untreated animal.

Several methods well known in the art can be used to detect apoptotic cells. Such methods include light and electron microscopy to detect morphological changes that occur during apoptosis, flow cytometry or density gradient centrifugation to detect characteristic cell shrinkage and increased granularity, assessment of membrane integrity using dyes such as trypan blue, ethidium bromide and acridine orange, measurement of the characteristic, nonrandom DNA fragmentation using techniques including agarose gel analysis, in vitro and in situ DNA end-labeling, PCR analysis, comet assays and ELISA systems, the detection of the activity of a caspase from the caspase family of protease enzymes that are activated during apoptosis, the detection of a phosphatidylserine binding protein such as annexin V, measurement of tissue transglutaminase activity, and measurement of calcium ion flux (Promega Notes, “Technically Speaking-Detecting Apoptosis,” 69:2 (1998)).

A variety of animal models of apoptosis also are well known in the art. Such animals can be used to determine if modulating the localization of a BAG polypeptide in a cell can alter a phenotype associated with an inappropriately large number of cells being in an apoptotic state. Axotomy-induced neuronal death in rat brain is an established model for studying apoptosis. A unilateral lesion of the visual cortex in the rat brain results in extensive neuronal cell death in the lateral geniculate nucleus (LGN) ipsilateral to the lesion (Moravec, R. and Riss, T. Promega Notes 68:13 (1998) and Agarwala, S. and Kalil, R. J. Comp. Neurol. 392:252 (1998)). Neurons in the LGN are axotomized by the lesion, which results in their atrophy and death at a precise time following induction of the lesion. The time course of this axotomy-induced cell death demonstrates that, at 3-days post-lesion, only 5% of the neurons in the ipsilateral LGN have perished; however, between days 3 and 7 after the lesion, extensive neuronal death occurs. Approximately two-thirds of the neurons in the dorsal ipsilateral LGN undergo cell death during this time. In addition to this predictable and well-documented animal model, other animal models are available to those skilled in the art. A variety of methods for detecting apoptotic cells, such as those described above, can be used to determine the number of apoptotic cells contained in untreated animals and in those treated to modulate the localization of BAG in cells of an animal model of apoptosis.

Expression of a BAG polypeptide at a particular subcellular localization can correlate with the differentiation state of a cell. For example, expression of BAG1 in the nucleus of mouse neuronal cells correlates with a differentiating state, as described in Example IV, Therefore, the invention provides a method for determining the differentiation state of a cell. The method involves (a) measuring an amount of BAG polypeptide at a subcellular location in a cell; (b) comparing the measured amount of BAG polypeptide to a reference amount of BAG polypeptide indicative of a particular differentiation state; and (c) identifying the differentiation state of the cell.

The amount of BAG polypeptide at a subcellular location in a cell, such as the nucleus or cytoplasm of a cell, can be determined qualitatively or quantitatively as described herein above, as described in Example IV, and by other well-known methods.

A reference amount of BAG polypeptide indicative of a particular differentiation state can be determined empirically for a particular cell type, for example, by measuring the amounts of BAG polypeptide contained in the cytoplasm and nucleus of the cell type in an undifferentiated and differentiated state, or in various state of differentiation. States of differentiation can include an undifferentiated state and various states of partial, immature, cell type-specific, cell stage-specific, or a differentiation state induced by a particular stimulus, incomplete differentiation, and a differentiated state. A cell in any differentiation state can be used to determine a reference amount of BAG polypeptide indicative of a particular differentiation state.

A differentiation state of a cell can be determined by identifying a reference amount of BAG polypeptide indicative of a particular differentiation state that is most similar to a measured amount of BAG polypeptide at a subcellular location in the test cell. For example, in a particular cell type, a differentiated state can be indicated by the presence of BAG polypeptide in the nucleus, but not in the cytoplasm, while an undifferentiated state can be indicated by the presence of BAG polypeptide in the cytoplasm, but not in the nucleus, or the reverse. Therefore, a measurement of the amount of BAG polypeptide in a subcellular location, such as the nucleus or cytoplasm, can be used to assess the differentiation state of the cell.

A measurement of the amount of BAG polypeptide in a second subcellular location can also be performed, and the amounts of BAG polypeptide in the two locations can be compared to reference levels of BAG polypeptide for a particular cell type. Measurements of amounts of BAG polypeptide at two or more subcellular locations can be absolute amounts, relative to a standard, or can be expressed as ratios, for example as a ratio of nuclear to cytoplasm BAG polypeptide amount, or a ratio of cytoplasm to nuclear BAG polypeptide amount. Reference differentiation stages also can be indicated by ratios of nuclear to cytoplasm BAG polypeptide content.

Further, the measured amounts of BAG polypeptide at first and second subcellular locations, such as the cytoplasm and nucleus, can be compared to predetermined amounts of BAG polypeptide at first and second subcellular locations. Such predetermined amounts of BAG polypeptide can be determined by measuring amounts of BAG polypeptide at first and second subcellular locations of a reference cell, or population of reference cells, in a particular differentiation state.

In addition, because subcellular location of a BAG polypeptide can be altered over time, as a cell progresses to a particular differentiation state, the amount of BAG polypeptide at a first and second location can be determined at different time points. A difference in the amounts of BAG polypeptide at first and second subcellular locations at different time points can be indicative of a differentiation state of the cell. For example, in a differentiating cell, the ratio of nuclear to cytoplasmic BAG polypeptide amount at an early time point could be high, indicating that BAG polypeptide location is mostly cytoplasmic, while the ratio of nuclear to cytoplasmic BAG polypeptide amount at a later time point, when the cell is fully differentiated, could be low, indicated that BAG polypeptide location is mostly nuclear, or the reverse.

The invention provides a method for identifying an agent that alters cell differentiation. The method involves (a) measuring an amount of BAG polypeptide at a subcellular location in a cell in the presence and absence of a candidate agent; and (b) identifying an agent that alters the amount of BAG polypeptide at the subcellular location, the agent being an agent that alters cell differentiation.

Once identified, an agent that alters cell differentiation can be used in methods for differentiating cells, such as precursor cells, including stem cells and neuronal precursor cells, as described above, or in another application of the methods of the invention for promoting differentiation, reducing proliferation or suppressing apoptosis.

An agent that alters the amount of BAG polypeptide at a cellular location can alter, for example, the amount of BAG polypeptide in the nucleus or cytoplasm. The alteration can be an increase or decrease in the amount of BAG polypeptide in a cellular location, such as the nucleus or cytoplasm. Such alteration can have an effect on the differentiation, proliferation or apoptotic state of the cell.

An agent that alters the amount of BAG polypeptide at a cellular location can include small organic molecules, nucleic acids, and polypeptides such as those derived from combinatorial and random libraries, and antibodies, including single chain antibodies (scFv), variable region fragments (Fv or Fd), Fab and F(ab)₂.

For use in the methods of the invention, cells can be obtained from a variety of mammals including, for example, mice, cows, primates and humans by methods well known in the art. For example, murine stem cells can be isolated from a mouse as described in Forrester et al., Proc. Natl. Acad. Sci. USA 88:7514-7517 (1991) or Bain et al., Devel. Biol. 168:342-357 (1995). Briefly, two-stage cell embryos can be isolated from fertilized female mice about 45 hours after injection with human chorionic gonadotropin. The two blastomeres can be fused by electrical impulse and cultured in M16 medium until the four cell stage is reached. ES cells can be grown on gelatin coated tissue culture flasks in DMEM (Dulbeco's modified Eagle's medium) containing high glucose and 1-glutamine (BRL) supplemented with 10% fetal bovine serum, 10% newborn calf serum, nucleosides stock, 1000 units/ml leukemia inhibitory factor, and 0.1 mM 2-mercaptoethanol.

Embryonic stem cells can be isolated from primates as described in Thomson (U.S. Pat. No. 5,843,780). Briefly, blastocysts can be removed from fertilized female monkeys 6-8 days after onset of ovulation, treated with pronase (Sigma) to remove the zona pellucida, rabbit anti-rhesus monkey spleen cell antiserum (for blastocysts from rhesus monkeys) and guinea pig complement (Gibco BRL), and washed in DMEM. The inner cell mass (ICM) can be removed from the lysed blastocyst with a pipette and plated on mouse gamma radiation inactivated embryonic fibroblasts. After 7 to 21 days the ICM derived masses can be removed with a micropipette, treated with 0.05% trypsin-EDTA (Gibco BRL) and 1% chicken serum, and replated on embryonic feeder cells. Colonies demonstrating ES morphology, characterized by compact colonies with a high nucleus to cytoplasm ratio and prominent nucleoli, can then be split as described above. The ES cells can be split by trypsinization or exposure to Dulbeco's phosphate buffered saline containing 2 mM EDTA every 1-2 weeks when cultures become dense.

Embryonic stem-like cells also can be isolated from cows as described in Cibelli et al., Nat. Biotech. 16:642-646 (1998). Briefly, oocytes can be removed from freshly slaughtered cows and placed in maturation medium M199 (Gibco), 10% fetal calf serum (FCS), 5 ug/ml bovine leutinizing hormone (Nobl) and 10 ug/ml pen-strep (Sigma) for 22 hours at 38.5° C. Oocytes can then be fertilized in vitro and cultured on mouse embryonic fibroblast feeder layers and CR2 with 6 mg/ml BSA until they reach the blastocyst stage. ES cells can be isolated from the blastocyst by mechanical removal of the zona pellucida and trophoblast with a 22 gauge needle and placed under mouse embryonic fibroblast feeder layers for one week. A small colony of the resulting cell mass can be removed and cultured on top of gamma irradiation inactivated mouse embryonic fibroblast feeder layer as cultures become dense.

Embryonic stem cells can be isolated from human blastocysts as described in Reubinoff et al., Nat. Biotech. 18:399-404 (2000). Briefly, fertilized oocytes can be cultured to the blastocyst stage and the zona pellucida digested by pronase (Sigma). The inner cell mass can be removed by immunosurgery with anti-human serum antibody (Sigma) and exposure to Guinea pig complement (BRL), and cultured on a mitomycin C mitotically inactivated mouse embryonic feeder cell layer in DMEM (BRL) supplemented with 20% fetal bovine serum (FBS, Hyclone) 0.1 mM 2-mercaptoethanol, 1% non essential amino acids, 2 mM glutamine, 50 units/ml penicillin and 50 ug/ml streptomycin (BRL)and 2,000 units/ml recombinant leukemia inhibitory factor. Cell mass clumps can be removed with a micropipette and replated on fresh feeder layer every six to eight days.

Neuronal precursor cells can be isolated from different areas of the brain (see, for example Vescovi et al., Brain Pathol 9:569-98 (1999) for a review, and Kim et al., Int. J. Dev. Neurosci. 19:631-8 (2001), and from cultured cells (see, for example, Guan et al., Cell Tissue Res 305:171-6 (2001)). A cell can be freshly obtained when convenient, or can be cultured, cryopreserved, or otherwise stored for a period of time prior to use.

A cell to be differentiated using a method of the invention can be contained within a tissue or other heterogeneous cell population, within a homogeneous cell population, or can be an isolated cell. When the cell to be differentiated is contained in a heterogenous cell population, expression of a BAG polypeptide can be selectively induced in the desired cell type. Methods for selective expression of polypeptides are well known to those skilled in the art and exemplary methods are described below. Alternatively, expression of a BAG polypeptide can be induced in multiple cell types.

When a cell is obtained from a tissue or other heterogenous cell population, a separation method can be used to isolate a desired cell type from other cell types. Such methods are well known to those skilled in the art and are described, for example, in Cell Separation Science and Technology, eds. D. S. Kompala and P. W. Todd, ACS Symposium Series, vol. 464 (1991). To confirm that an isolated cell type has a phenotype consistent with the desired cell type, morphological, biochemical or genetic markers can be used. Appropriate neuronal cell type markers are known to those skilled in the art.

The following examples are intended to illustrate but not limit the present invention.

### EXAMPLE I

### Over-expression of Bag1 in Neuronal CSM 14.1 Cells

This example shows that mouse Bag1 can be exogenously expressed in CSM 14.1 neuronal cells.

A plasmid was constructed for expression of flag-mouse Bag (mBag1) under control of the neuron-specific enolase (NSE) promoter. First, the 1.8 kb 5′-fragment of the rat NSE-promoter (Forss-Petter et al., Neuron 5:187-197 (1990)) was subcloned into pBSKI1 using the EcoRI and HindIII restriction sites. In a second step, this plasmid was digested with BamHI and HindIII. The resulting NSE-promoter containing fragment was used to replace the HSV-TK promoter in PRL-TK (Promega, Madison, Wis.) between the BglII and HindIII sites. The resulting construct containing a luciferase gene under the control of the NSE promoter served as control vector. Subsequently, flag-mBag1 was inserted at the NheI and Not1 sites from pCI-flag-mBag1 (Takayama et al., Cell 80:279-284 (1995)).

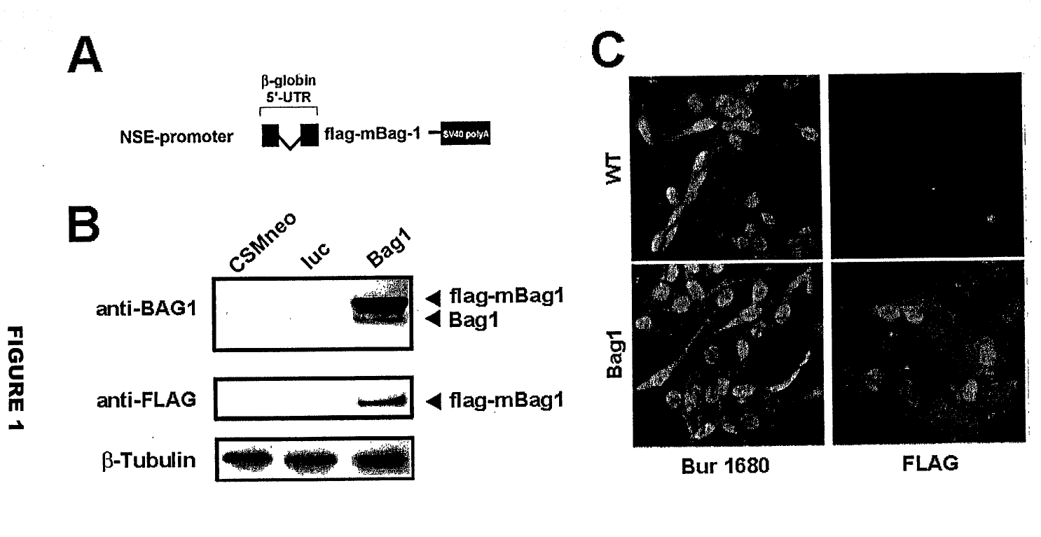

To over-express Bag1 in a neuronal cell line, rat nigro-striatal CSM14.1 cells, (Zhong et al., Proc. Natl. Acad. Sci. USA 90:4533-4537 (1993)) were transfected with a plasmid containing flag-tagged mouse p29 Bag1 driven by the neuron-specific enolase (NSE) promoter (FIG. 1A).

Cells were maintained in Dulbecco's modified Eagle's medium (DMEM) supplemented with 10% FBS (fetal bovine serum), 1 mM L-glutamine, 100 U/ml penicillin, and 100 ug/ml streptomycin sulfate either in 32° C. at permissive or 39° C. at non-permissive temperature (Zhong et al., supra). For stable transfection, 50-70% confluent CSM cells in 6-well plates were incubated with Gene Porter II according to the supplier's protocol (Gene Therapy Systems, San Diego, Calif.) in serum-free medium containing 3 ug of plasmid DNA representing a 5:1 ratio of specific plasmid:puromycin-resistance plasmid (pBabe-puro). After 3 h at 37° C., serum-containing medium was added and the cells were incubated overnight. Finally, the transfection agent was replaced by 10% serum-containing medium and the cells were transferred to 32° C. Selection with 4 ug puromycin in complete medium was started the next day. After 5 days, 0.5 cells per well were seeded in 96-well plates with selection medium, and 3 to 4 weeks later wells containing single clones were identified by light microscopy and the cells transferred to larger plates for expansion and further processing.

Expression of mouse Bag1 was determined by immunoblot analysis of lysates (20 ug per lane) from wild-type (CSMneo), empty vector-transfected (luc) and Bagl-transfected (Bagl) CSM 14.1 cells prior to isolating clones, using Anti-Bag1 antiserum Bur 1680 developed using ECL reagent on nitrocellulose membranes. Flag-mBag1, which migrates in gels at a slightly higher molecular weight than endogenous Bagl, was strongly expressed (FIG. 1B). Out of 30 clones tested, 12 were positive for over-expression of Bagl. Two clones (# 15 and 20) were chosen for further analysis. In FIG. 1B, the plasmid-derived flag-mBag1 and endogenous Bag1 are indicated by arrowheads. Via co-transfection, pBabePUR0 containing a puromycin resistance gene was introduced together with the Bag1 construct as selection marker. A plasmid containing a NSE-driven luciferase gene served as control vector.

These results indicate that mouse Bag1 can be over-expressed in CSM 14.1 neuronal cells.

### EXAMPLE II

### Localization of Bagl Protein in CSM 14.1 Cells

This example shows that over-expression of Bag1 in CSM 14.1 neuronal cells results in increased levels of nuclear-localized Bag1 polypeptide.

To determine the intracellular location of Bag1 in CSM14.1 neuronal cells, immunofluorescence microscopy analysis of stably-transfected cells was performed using two antibodies raised against different epitopes of Bagl. Prior to and after selection of single cell clones, gene expression was determined by immunoblot analysis employing antibodies against Bag1 (Bur 1680 and 1735) and Flag. Immunofluorescence microscopy was performed employing polyclonal Bag1 antiserum or monoclonal anti-FLAG antibody, followed by FITC-labeled secondary antibody (400× magnification).

In FIG. 1C, the upper row shows control-transfected cells (WT), revealing nuclear and cytoplasmic expression of endogenous Bagl. No signal was detected with FLAG antibody. In contrast, Bag1 immunoflourescence in stable transfectants (lower row, Bagl) is strongly increased with predominantly nuclear localization of flag-mBag1 revealed by staining with the anti-FLAG antibody. Compared to levels of endogenous Bag1 expression in CSM14.1 cells, an increase in nuclear immunofluorescence in cells over-expressing Bag1 was observed (FIG. 1C). In contrast, staining with preimmune serum and rabbit IgG produced no immunofluorescence, demonstrating the specificity of these results (not shown).

These results show that Bag1 polypeptide localizes primarily to the nucleus when over-expressed in CSM14.1 neuronal cells.

### EXAMPLE III

### Bagl Inhibits Cell Death after Serum Starvation

To determine the effect of over-expression of Bag1 on serum starvation of CSM14.1 cells, the cells were starved and indicia of cell death were observed. Serum starvation induces death of CSM14.1 cells maintained at 39° C. (Zhong et al., Proc. Natl. Acad. Sci. USA 90:4533-4537 (1993)). Prior to serum deprivation, cells were maintained at ‘non-permissive’ temperature for 2-3 days. Cell death was induced by serum deprivation as published previously (Zhong et al., supra). 10⁵ cells were plated in 6-well plates and maintained in 39° C. Prior to serum deprivation, cells were washed 3 times in serum-free medium. Cell death was assessed by Trypan Blue exclusion after 24 hrs, 2, 3, and 4 days in serum starvation. Experiments were repeated 6 times. Two clones (15 and 20) expressing comparable levels of flag-mBag1 were analyzed (see insert).

FIG. 2 shows that after 2 days without serum, approximately 40% of wild-type cells were dead. This death can be largely attributed to apoptosis, since cells displayed fragmented nuclei (as visualized by DAPI staining) and could be protected by treatment with the broad-spectrum caspase inhibitor z-VAD-fmk. As shown in FIG. 2, stable Bag1 over-expression significantly prevented cell death measured at 24 and 48 hours after serum deprivation (p<0.01), while transfection with control vector (CSMluc) did not. Both of the stably-transfected clones tested, Bag15 and Bag20, which express the transgene at comparable levels (see insert in FIG. 2), displayed substantially reduced cell death rates (−50%) when compared to control-transfected cells. Values marked ** were found to be statistically significant when compared to CSMneo (p<0.01).

A comparison in the ability of Bag1 over-expressing CSM 14.1 cells and Bcl-2 over-expressing CSM 14.1 cells to overcome serum-induced cell death was performed. As shown in FIG. 2, Bcl-2 was more effective than Bag1 in inhibiting cell death following serum deprivation (FIG. 2).

### EXAMPLE IV

### Bagl Induces Differentiation of Csm 14.1 Cells

This example shows that CSM14.1 cells over-expressing Bag1 developed a more differentiated phenotype.

Bag1 over-expressing cells were observed to have altered morphology, even when cultured at the ‘permissive’ temperature of 32° C. In contrast to the small and round shape of wild-type cells, Bag1 over-expressing cells are larger and display a more polarized shape.

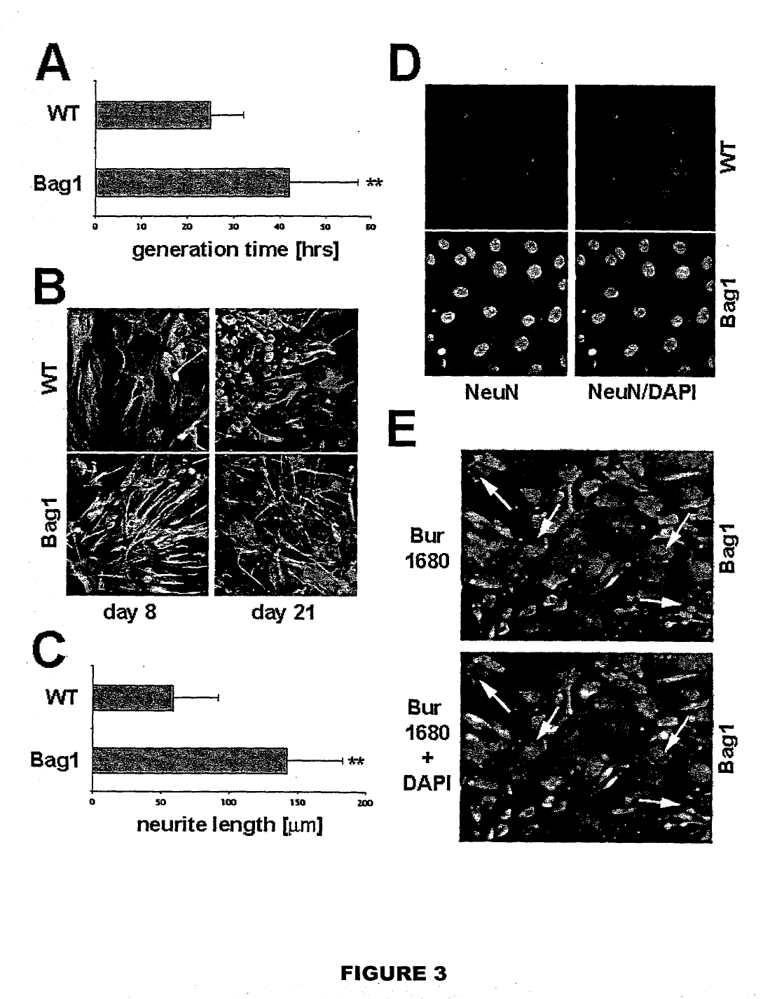

The rate of proliferation in Bag1 over-expressing cells was examined for cells grown at permissive and non-permissive temperatures. Generation time was assessed by counting cell numbers during logarithmic growth at permissive temperature (32° C.) over one week in 24-well plates. 10⁴ cells were plated and daily cell counts were performed by Trypan Blue exclusion assay for 1 week. Data were averaged for wild-type and control-transfected (WT) as well as two independent Bag1 over-expressing clones (Bagl). Average generation time (mean+SD; n=3) was significantly higher in Bag1 over-expressing cells (p<0.01). A difference in the generation times of the respective cell lines at 32′C (p<0.01) was observed. Cells over-expressing Bag1 were less proliferative, with an average generation time of 42 hrs, whereas wild-type CSM14.1 and empty vector transfected cells doubled on average every 25 hrs (FIG. 3A).

Using immunostaining, cell morphology of CSM14.1 cells grown at the non-permissive temperature of 39° C. (where the SV40 large T-antigen is inactive) was observed at various time points after switching to 39° C. Immunostaining was performed using anti-Tubulin antibody (visualized with FITC-coupled secondary antibody). Cells were co-stained with DAPI. Photomicrographs show wild-type or control-transfected (upper row, WT), and Bag1 over-expressing cells (lower row, Bagl) on day 8 and 21 after switching to ‘non-permissive’ temperature (200× magnification).

FIG. 3B contrasts the morphology of wild-type and Bag1 over-expressing cells at two different times after switching to 39° C. After 8 days at 39° C., most wild-type and control-transfected CSM14.1 cells had an enlarged cell soma containing a big nucleus. However, few of these cells had started to extend axon-like processes. In contrast, in cultures of Bag1 over-expressing cells, almost all of the cells were smaller and assumed a more polarized shape. Virtually all of these cells had grown processes, which often aligned, forming bundles. The length of outgrowing neurites was assessed on day 8 at 39° C. in 3 independent experiments on randomly picked cells using ImagePro-Plus software. These measurements revealed that axon-like structures in Bag1 over-expressing cells had grown almost 3 times longer than processes in wild-type cells (p<0.01; FIG. 3C).

After extended periods at 39° C., the morphological differences became even more pronounced. After 21 days at non-permissive temperature, Bag1 over-expressing cells were characterized by striking arborization of processes and formation of dense nests of axon-like connections between cells. In contrast, cells in cultures of wild-type or control-transfected cells had a less differentiated appearance, with residual large cells and far fewer cellular processes (FIG. 3B).

To correlate these differences in morphological changes with markers of the neuronal phenotype, expression of neuronal differentiation-associated proteins was examined.

FIG. 3D shows WT (upper row) and Bag1 over-expressing (lower row) cells were fixed after 28 days culture at 39° C. and stained using FITC-labeled NeuN and DAPI (400× magnification). While NeuN staining in WT cells is absent or very faint, almost all nuclei in Bag1 over-expressing cells expressed the NeuN antigen.

After extended time at non-permissive temperature, weak Neurofilament-200 staining could be detected in cultures of either control- or Bag1 over-expressing CSM 14.1 cells. In contrast, positive staining for NeuN antigen (a marker of post-mitotic neuronal nuclei) was observed in most Bag-l over-expressing cells by day 21 at 39° C., while NeuN staining was very weak or completely absent in control CSM14.1 cells (FIG. 3D).

During neuronal differentiation at non-permissive temperature, a loss of nuclear Bag1 expression was observed. FIG. 3E illustrates localization of Bag1 in stably transfected CSM14.1 cells after 28 days at non-permissive temperature using polyclonal antiserum against Bag1 (Bur 1680). In contrast to the mainly nuclear location of flag-mBag1 at permissive temperature (compare FIG. 1C), most Bag1 over-expressing cells displayed an exclusively cytosolic location of Bag1, as revealed by double-labeling with DAPI (FIG. 3E). Staining with preimmune serum and control rabbit IgG served as a negative control.

For immunofluorescence, wild-type cells and those stably transfected with either empty vector or flag-mBag1 were trypsinized and seeded into chamber slides. Cells were either maintained in 32° C. or 39° C. until further processing. After various lengths of time, cells were washed in PBS and fixed in PBS containing 4% paraformaldehyde for 5 min at room temperature, followed by several washing steps in PBS. Permeabilization was performed in 0.3% Triton X-100/PBS for 5 min with subsequent preblocking in PBS containing 2% normal goat serum. Cells were incubated in blocking solution containing the following primary antibodies: Bur 1680 and 1735 (1:100), anti-Tubulin (1:2000), anti-Flag M5 (1:250), NeuN (Sigma, 1:50), and MAP-2 (Sigma, 1:100). After washing three times in PBS and incubation with FITC-conjugated secondary anti-mouse or anti-rabbit antibody (Dako, 1:50) for 2 hrs at room temperature, slides were covered with Vectashield mounting medium with or without 1.5 ug/ml 4,6-diamidino-2-phenylindole (DAPI) (Vector Laboratories, Burlingame, Calif.) and sealed with Cytoseal 60 mounting medium (Stephens Scientific, Kalamazoo, Mich.).

These results show that Bag1 expression in the nucleus of CSM14.1 cells correlates with a differentiated neuronal phenotype, whereas Bag1 expression in the cytoplasm of CSM14.1 cells correlates with an undifferentiated phenotype.

### EXAMPLE V

### Bal1 Over-Expression Induces MAPK-Pathway

This example shows the role of MAP kinases in differentiation of CSM14.1 cells over-expressing Bag1.

To determine levels of MAP kinases Erk 1 and Erk 2, lysates of wild-type (WT) and Bag1 over-expressing (Bag1) cells at different time-points after switch to 39° C. were subjected to SDS-PAGE and analyzed by immunoblot incubating the same membrane sequentially with phospho-Erkl/2, Hsp70, Bag1 and Erkl/2 antibodies. Bag1 over-expressing cells displayed markedly increased levels of phospho-Erkl/2.

Cell lysates were prepared at different times after switching cells to non-permissive temperature using RIPA buffer as described in (Krajewski et al., J. Neurosci. 15:6364-6376 (1995)). Proteins (20 μg per lane) were resolved by SDS-PAGE and transferred onto nitrocellulose membranes. After blocking with 5% skim milk, 2% bovine serum albumin (BSA) in TBST (10 mM Tris [pH7.5]; 142 mM NaCl; 0.1% Tween-20) at room temperature for 2 hrs, blots were incubated in the same solution with various primary antibodies including polyclonal antisera against Bag1 (Bur 1735 and Bur 1680;l:l000; see above), phospho-Erkl/2 and Erkl/2 (Cell Signaling; l:l000), as well as monoclonal antibodies against Flag (Sigma M2 or M5; 3 ug/ml) and Hsp70 (ABT; 1:5000), followed by horseradish peroxidase-conjugated anti-mouse or anti-rabbit IgG (Biorad) secondary antibodies. Bound antibodies were visualized using an enhanced chemiluminescence (ECL) detection system (Amersham).

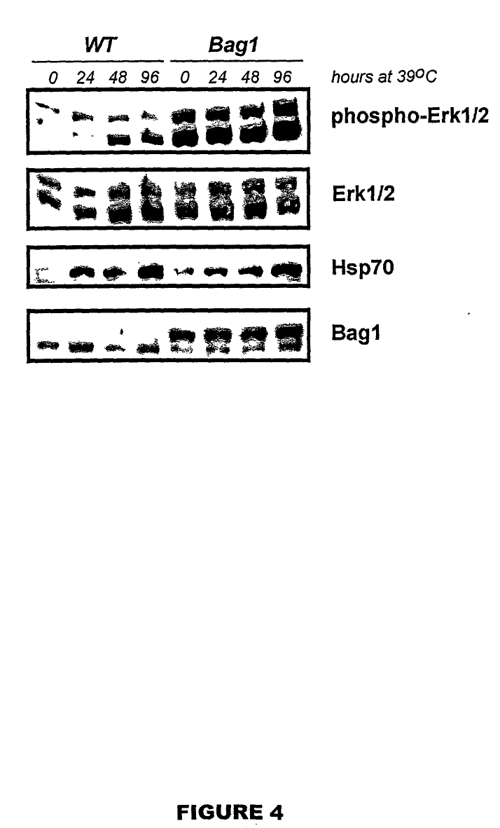

FIG. 4 shows immunoblot analysis of Erkl/2 phosphorylation, which correlates with Erkl/2 activity. Bag1 over-expressing cells showed increased levels of phospho-Erkl/2 at permissive temperature of 32° C. when compared to wild-type cells. These high levels of Erkl and 2 phosphorylation were maintained after switch to 39° C. In wild-type cells, only a modest increase in Erk-phosphorylation could be detected following temperature switch, while the expression of non-phosphorylated Erks did not change significantly in both cell types over time. Interestingly, the temperature switch induced increased levels of Hsp70 with similar kinetics in both wild-type and Bag1 over-expressing cells (FIG. 4).

These results show that MAP kinase activity is increased in Bag-l over-expressing CSM14.1 cells.

### EXAMPLE VI

### Bag1 Expression During Development of the Mouse Nervous System

This example shows Bag1 expression in the developing mouse nervous system.

The timing and distribution of Bag1 protein expression in the developing nervous system were assessed in paraffin sections derived from embryos and postnatal mice of the NMRI or FVB strains. All procedures were approved by the institutional animal care committee. Prenatal development was studied on a closely spaced series of mouse embryos at daily intervals from 6 days of gestation (E6) to postnatal day 4 (P4). Twenty-five mice were studied at weekly intervals after that time until adulthood. Mice were mated overnight, and the morning the vaginal plug appeared was designated as embryonal day 0.5 (E0.5). The day of birth was termed as postnatal day zero (PO). All embryos were taken from mice which had been killed by over-dose of carbon dioxide. At E4-9, the uterus was excised and fixed with the embryos in situ. For the later embryos, each embryo was dissected from the uterus, freed from the extra-embryonic membranes and immediately placed in the fixatives, either Bouin's fixative, or zinc-buffered formalin (Z-Fix; Anatech LTD, Battle Creek, Mich.). Immersion time varied from 2 days for early stages to 5-7 days for fetal and postnatal specimens. Altogether, tissue specimens from 58 embryos and 29 mice after birth have been paraffin-embedded according to routine procedures.

Dewaxed tissue sections were exposed to polyclonal antibodies and confirmed to be specific for Bag1. The sections were immunostained using a diaminobenzidine (DAB)-based detection method as described in detail, employing either an avidin-biotin complex reagent (Vector Laboratories, Burlingame, Calif.) or the Envision-Plus-Horse Radish Peroxidase (HRP) system (DAKO, Carpinteria, Calif.) using an automated immunostainer (Dako Universal Staining System) (33, 35). The dilutions of antisera typically employed were 1:3500 (v/v) for #1735, 1:5000 for #1680, and 1:2500 for #1702.

To verify specificity of the results, the immunostaining procedure was performed in parallel using preimmune serum or anti-Bag1 antiserum preadsorbed with 5-10 ug/ml of synthetic peptide immunogen.

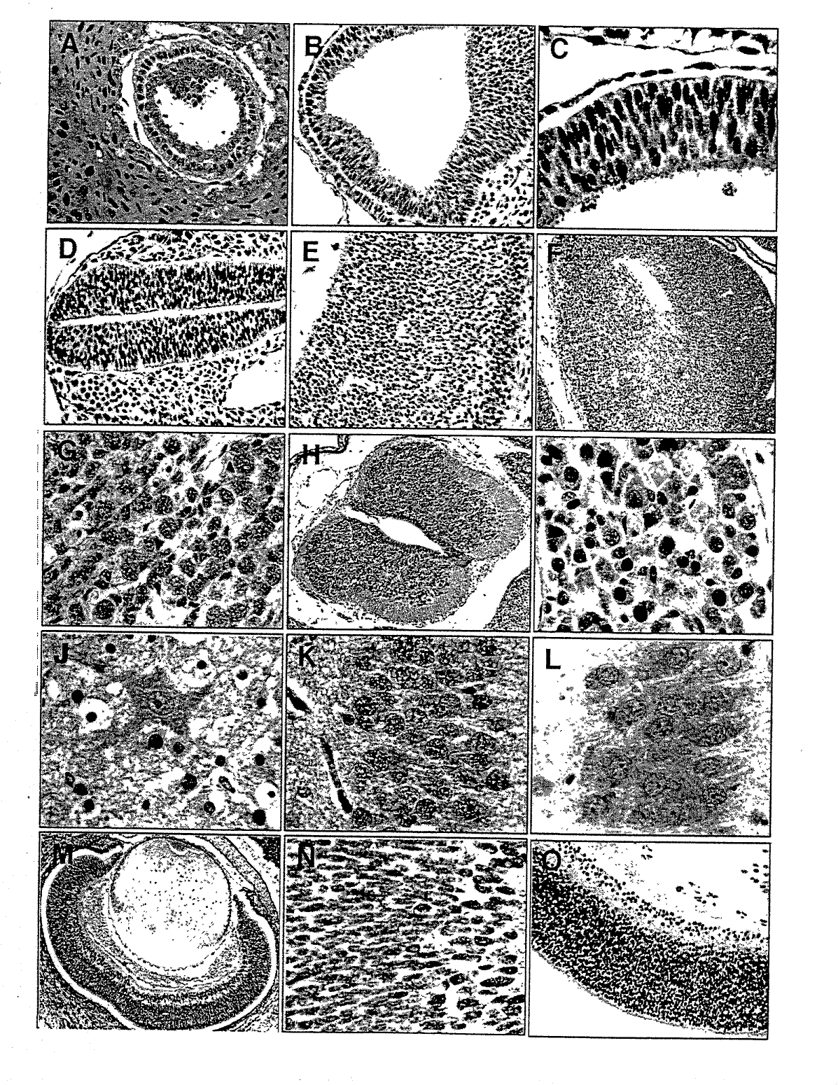

FIG. 5 shows representative photomicrographs of the analysis of Bag1 expression in the mouse nervous system. Antibody detection was achieved using a DAB-based chromogenic method (brown) and nuclei were counterstained with hematoxylin. All results presented in this figure were obtained using a rabbit polyclonal antibody raised against a synthetic peptide (BUR1735), but similar results were observed using alternative antibodies (BUR1680 and 1702, not shown). (A) At the onset of neurulation (E7.5-8), the primitive neural tube exhibited barely detectable levels of cytosolic Bag1 (magnification 250×). (B, C) At E8.5, Bag1 nuclear immunostaining appeared in the differentiating neuroblasts of the primary brain vesicles (magnification 250× and 1000×, respectively). (D) At E8, neuroblasts in the caudal part of neural tube exhibit high levels of Bag1 nuclear expression (magnification 250×). (E) Only occasional neuroblasts in the proliferative, periventricular matrix zone show Bag1 staining, whereas the differentiating neuronal cells in the mantle layer demonstrate strong Bag1 immunoreactivity (E11) (magnification 250×). (F) The majority of neuronal cells in hypothalamus and basal part of corpus striatum demonstrate intense nuclear staining and increasing cytoplasmic signal for Bag1 (E12) (magnification 150×). (G) At E12, strong nuclear immunoreactivity is evident in the differentiating sensory neurons of the dorsal root ganglia (magnification 400×). (H) In the spinal cord (E14.5), note the elevated nuclear Bag1 immunoreactivity in the ventral motor neurons, and a negligible amount of this protein in the dorsal part (magnification 80×). (I) An appearance of cytosolic immunostaining for this protein is noticed in the sympathetic trunk ganglia (E17) (magnification 400×). (J) A terminally differentiated motor neuron in the spinal cord is shown, demonstrating loss of the nuclear Bag1 staining at E17 (magnification 1000×). High nuclear Bag1 expression in neurons of CA3 hippocampal sector in the later fetal stages (E17) (K; magnification 1000×), is down-regulated in their adult counterparts (L; magnification 1000×). (M, N) At E17, the outer-most, proliferating layer of primitive neuroepithelial cells in the retina is mostly immunonegative. The neuroblasts in the inner layer, which will differentiate into the ganglion cells, contain high levels of nuclear Bag1 (magnification 100× and 1000×, respectively). (O) Control immunostaining using anti-Bag1 antiserum preadsorbed with 5 ug/ml of synthetic peptide immunogen showed negative staining of the neural retina (magnification 250×).

At the onset of neurulation (E7.5-8), neural folds in the cephalic region contained only trace levels of Bag1 immunostaining. A single layer of pseudostratified columnar epithelium, constituting the neuroepithelium of the primitive neural tube, exhibited barely detectable cytosolic Bag1 staining (FIG. 5A). With the formation of the three primary brain vesicles at E8.5-9.5, Bag1 nuclear staining appeared and was located predominantly in the differentiating neuroblasts of the rhombencephalon and mesencephalon, and at lower levels in the prosencephalon (FIGS. 5B, C). Numerous neuroblasts in the caudal part of the neural tube (which subsequently differentiates into the spinal cord) contained moderate levels of Bag1 nuclear labeling (FIG. 5D).

At a later stage of neural differentiation (El0-ll), when the division of the primitive neural tube into three concentric layers occurs, neuroblasts in proliferative, periventricular matrix zone were consistently negative for Bag1. An obvious gradient of Bag1 expression appeared at this stage, associated with differentiation of the peripherally migrating progeny of ventricular neuroblasts to the intermediate mantle layer. The cells with early features of neuronal differentiation in the mantle layer, which gives rise to the gray matter of the central nervous system, demonstrated strong nuclear Bag1 immunostaining (FIG. 5E).

At E12, when the various regions of the brain are more clearly defined, roughly half of the migrating postmitotic neurons evidenced not only intense nuclear staining but also increasing cytoplasmic Bag1 immunoreactivity (FIG. 5F) in the hypothalamus, thalamus, and corpus striatum (the major derivatives of the diencephalon). At this time, a significant increase of Bag1 immunoreactivity also became apparent in the developing peripheral nervous system. Strong nuclear labeling was evident in the differentiating sensory neurons of the dorsal root ganglia (FIG. 5G), and in the segmental ganglia along sympathetic trunks. The cranial ganglia, such as the facial (VII), acoustic (VIII), and the glossopharyngeal (IX) ganglion complexes revealed a similar distribution pattern.

By El3-14 in the central nervous system of developing embryos, differentiating neuroblasts in the neopallial cortex (which is formed as cells from the mantle layer of the telencephalic vesicles migrate into the overlying marginal zone to constitute in due course the outer grey layer of the cerebral hemispheres) contained moderate levels of cytosolic Bag1, whereas the intensity of the nuclear signal had greatly declined. In the forming hippocampal plate, nuclear Bag1 immunoreactivity remained only in the rare residual migrating neuroblasts throughout most of the thickness of the plate. This trend toward diminishing nuclear and increasing cytosolic Bagi immunostaining in more differentiated neurons continued throughout the remaining nervous system development in multiple regions of the brain and peripheral nervous system, including the spinal cord (FIGS. 5H, J), sympathetic trunk ganglia (FIG. 5I), olfactory bulb, pyramidal neurons of the CA3-CA4 sector of the hippocampus (FIGS. 5K, L), diencephalon, mesencephalon and rhombencephalon as well as the retina (FIGS. 5M, N). Control stainings performed with preimmune serum or using anti-Bag1 antibody that had been pre-adsorbed with Bag1 protein or peptide antigen confirmed the specificity of these results (FIG. 5O).

These results show that during late fetal life and into adulthood, selected types of neurons expressed Bag1, which was predominantly localized to the cytosol.

### EXAMPLE VII

### Generation of Bag1 Antisera

Polyclonal antisera for Bag1 were generated in rabbits using synthetic peptides or GST-fusion protein immunogens. A peptide (NH2-CNERYDLLVTPQQNSEPVVQD-amide) corresponding to residues 26-45 of the mouse Bag1 protein, was synthesized with an N-terminal cysteine appended to permit conjugation to maleimide-activated carrier proteins KLH and OVA (Pierce, Inc.), as described previously (Krajewski et al., Am. J. Pathol. 145:1323-1333 (1994)). This peptide conjugate was used to generate a polyclonal antiserum (#1735) in rabbits (Takayama et al., Cancer Res. 58:3116-3131 (1998)). An additional anti-Bag1 serum (#1680) was generated in rabbit using a GST-mouse Bag1 (8-219) fusion protein (Takayama et al., Cell 80:279-284 (1995)). The generation and characterization of a rabbit anti-mouse Bag1 antiserum targeted against amino acids 204-219 (#1702) have been described (Takayama et al., Cell 80:289-284 (1995)).

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention.

What is claimed is:

1. A method for promoting cell differentiation, comprising modifying a cell to increase expression of a BAG polypeptide, wherein said BAG polypeptide promotes differentiation of said cell.

2. The method of claim 1, wherein said cell is a neuronal cell, stem cell or neural progenitor cell.

3. The method of claim 1, wherein said modification comprises introducing a recombinant nucleic acid to said cell, the recombinant nucleic acid encoding said BAG polypeptide.

4. The method of claim 1, wherein said modification comprises adding an inducing agent to said cell, wherein said inducing agent increases expression of a nucleic acid encoding said BAG polypeptide.

5. The method of claim 1, wherein said cell is a human cell.

6. The method of claim 1, wherein said cell is a non-human mammal cell.

7. The method of claim 1, wherein said BAG polypeptide comprises a nuclear localized BAG polypeptide.

8. The method of claim 1, wherein said BAG polypeptide comprises Bag1.

9. A method for promoting cell differentiation, comprising modifying a cell to increase the amount of a nuclear localized BAG polypeptide, wherein said nuclear localized BAG polypeptide promotes differentiation of said cell.

10. The method of claim 9, wherein said cell is a neuronal cell, stem cell or neural progenitor cell.

11. The method of claim 9, wherein said modification comprises introducing a recombinant nucleic acid to said cell, the recombinant nucleic acid encoding said BAG polypeptide.

12. The method of claim 9, wherein said modification comprises adding an inducing agent to said cell, wherein said inducing agent increases expression of a nucleic acid encoding said BAG polypeptide.

13. The method of claim 9, wherein said cell is a human cell.

14. The method of claim 9, wherein said cell is a non-human mammal cell.

15. The method of claim 9, wherein said BAG polypeptide comprises Bag1.

16. A method for reducing the rate of cell proliferation, comprising modifying a cell to increase the amount of a nuclear localized BAG polypeptide, wherein said nuclear localized BAG polypeptide inhibits proliferation.

17. The method of claim 16, wherein said cell is a neuronal cell, stem cell or neural progenitor cell.

18. The method of claim 16, wherein said modification comprises introducing a recombinant nucleic acid to said cell, the recombinant nucleic acid encoding said BAG polypeptide.

19. The method of claim 16, wherein said modification comprises adding an inducing agent to said cell, wherein said inducing agent increases expression of a nucleic acid encoding said BAG polypeptide.

20. The method of claim 16, wherein said cell is a human cell.

21. The method of claim 16, wherein said cell is a non-human mammal cell.

22. The method of claim 16, wherein said BAG polypeptide comprises Bag1.

23. A method for suppressing apoptosis, comprising modifying a cell to increase the amount of a nuclear localized BAG polypeptide, wherein said nuclear localized BAG polypeptide suppresses apoptosis.

24. The method of claim 23, wherein said cell is a neuronal cell, stem cell or neural progenitor cell.

25. The method of claim 23, wherein said odification comprises introducing a recombinant nucleic cid to said cell, the recombinant nucleic acid encoding said BAG polypeptide.

26. The method of claim 23, wherein said modification comprises adding an inducing agent to said cell, wherein said inducing agent increases expression of a nucleic acid encoding said BAG polypeptide.

27. The method of claim 23, wherein said cell is a human cell.

28. The method of claim 23, wherein said cell is a non-human mammal cell.

29. The method of claim 23, wherein said BAG polypeptide comprises Bag1.

30. A method for identifying the differentiation stage of a cell, comprising:
(a) measuring an amount of BAG polypeptide at a subcellular location in a cell;
(b) comparing said measured amount of BAG polypeptide to a reference amount of BAG polypeptide indicative of a particular differentiation stage; and
(c) identifying the differentiation stage of said cell.

31. The method of claim 30, wherein said subcellular location is the nucleus.

32. The method of claim 30, wherein said subcellular location is the cytosol.

33. The method of claim 30, wherein said amount of BAG polypeptide is determined by an immunological method.

34. The method of claim 30, wherein said BAG polypeptide comprises Bag1.

35. The method of claim 30, further comprising:
(d) measuring an amount of BAG polypeptide at a second subcellular location of a cell.

36. The method of claim 35, wherein step (b) further comprises, comparing said measured amounts of BAG polypeptide at said second and first subcellular location to predetermined amounts of BAG polypeptide at said first and second subcellular location indicative of a particular differentiation stage.

37. A method for identifying an agent that alters cell differentiation, comprising:
(a) measuring an amount of BAG polypeptide at a subcellular location in a cell in the presence and absence of a candidate agent; and
(b) identifying an agent that alters the amount of BAG polypeptide at said subcellular location, said agent being an agent that alters cell differentiation.

38. The method of claim 37, wherein step (b) comprises identifying an agent that modulates the amount of BAG polypeptide in a cell nucleus.

39. The method of claim 38, wherein step (b) comprises identifying an agent that increases the amount of BAG polypeptide in said nucleus.

40. The method of claim 38, wherein step (b) comprises identifying an agent that decreases the amount of BAG polypeptide in said nucleus.

41. The method of claim 37, wherein step (b) comprises identifying an agent that modulates the amount of BAG polypeptide in a cell cytosol.

42. The method of claim 41, wherein step (b) comprises identifying an agent that increases the amount of BAG polypeptide in said cytosol.

43. The method of claim 41, wherein step (b) comprises identifying an agent that decreases the amount of BAG polypeptide in said cytosol.
